(19) United States
(12) Patent Application Publication
(10) Pub. No.: US 2008/0119552 A1
(43) Pub. Date: May 22, 2008

(54) MATRIX-EMBEDDED COMPOSITIONS HAVING ORGANIC ACIDS AND FATTY ACIDS

(75) Inventor: Felipe Navarro, O'Fallon, MO (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: NOVUS INTERNATIONAL INC., St. Louis, MO (US)

(21) Appl. No.: 11/939,019

(22) Filed: Nov. 13, 2007

Related U.S. Application Data
(60) Provisional application No. 60/866,346, filed on Nov. 17, 2006.
(60) Provisional application No. 60/866,348, filed on Nov. 17, 2006.

(51) Int. Cl. A61K 47/06; A23D 7/005; A61K 31/19; A61K 47/12; A61K 31/192; A61K 31/20

(52) U.S. Cl. 514/558; 426/601; 514/557; 514/568; 514/772; 514/784

(57) ABSTRACT

The invention generally provides matrix-embedded compositions having organic acids and fatty acids. The compositions may be administered to an animal to deliver intact organic acids and fatty acids to the animal's small intestine. The invention also provides monograstric feed rations comprising the matrix-embedded compositions.

### CROSS-REFERENCE TO RELATED APPLICATIONS

This application claims priority to U.S. Provisional Application No. 60/866,348 filed on Nov. 17, 2006, which is hereby incorporated by reference in its entirety.

### FIELD OF THE INVENTION

The invention generally provides matrix-embedded compositions having organic acids and fatty acids. The compositions may be administered to an animal to deliver intact organic acids and fatty acids to the animal's small intestine.

### BACKGROUND OF THE INVENTION

There is a growing demand for nutritional supplements or liquid food supplements that provide energy, nutrients, vitamins, and/or minerals to humans and animals. Such supplements have traditionally been given to infants, the elderly, or severely ill patients to provide life-saving nutrition. Nutritional supplements may also be used by athletes to boost strength and performance or by ordinary persons with hectic lifestyles to provide a balanced diet. Furthermore, they may be given to companion animals to meet their nutritional needs or to agricultural animals to promote growth and health.

Nutritional supplements may contain a quick energy source in the form of fatty acids or triglycerides (glycerol esters of fatty acids) rather than glucose or another form of sugar. Short chain (C₂-C₆) fatty acids are typically generated in the large intestine by microbial fermentation of non-digestible starches or soluble fiber. Short chain fatty acids are readily absorbed and oxidized for energy or used to generate ATP. The addition of short chain fatty acids or short chain triglycerides to a nutritional supplement enables these fatty acids to be absorbed earlier in the intestinal tract. Medium chain (C₈-C₁₂) triglycerides are regularly added to infant formulas because breast milk is highly enriched with these molecules. Medium chain triglycerides are digested and absorbed much more quickly than long chain triglycerides, and thus provide a quick source of energy. Both short chain and medium chain fatty acids acidify the intestine, thereby, providing antimicrobial activity by restricting the growth and activity of less beneficial bacterial species. One problem associated with the addition of fatty acids to a food supplement, however, is that the fatty acid may be degraded in the harsh acidic environment of the stomach.

Several types of encapsulated products have been utilized to protect organic acids so that they remain intact upon arrival in the small intestine. Encapsulated products typically consist of a protective coating that completely surrounds or “encapsulates” the organic acid. One drawback with encapsulation technology, however, is that the protective coating can be compromised in the stomach. In turn, the compromised coating causes the release of all of the organic acid in the stomach as opposed to the small intestine.

While it is well established that nutrition supplements may beneficially contain fatty acids, there is a need for a mechanism to deliver sufficient quantities of these nutrients in an intact state to the intestine for ready absorption.

### SUMMARY OF THE INVENTION

One aspect of the invention provides a composition embedded in a lipid matrix. The composition comprises an organic acid and fatty acid having from four to twelve carbon atoms.

Another aspect of the invention encompasses a method for providing an organic acid and a fatty acid having from four to twelve carbon atoms to a monogastric animal. The method comprises administering to the monogastric animal a composition comprising an organic acid and a fatty acid embedded in a lipid matrix. Typically, the organic acid and fatty acid are not substantially released from the matrix until the composition enters the monogastric animal's small intestine.

Yet another aspect of the invention provides a monogastric animal feed ration. The feed ration comprises grain, crude protein, crude fat, and a composition comprising an organic acid and a fatty acid embedded in a lipid matrix.

### BRIEF SUMMARY OF THE DRAWINGS

FIG. 1 depicts a graph illustrating the initial weight of the piglets in each diet group, the final weight of the piglets in each diet group, and the weight development of the piglets in each diet group.

FIG. 2 depicts a graph illustrating the daily weight gain of the piglets in each diet group.

FIG. 3 depicts a graph illustrating the feed conversion for the piglets in each diet group.

FIG. 4 depicts schematics illustrating two techniques for protecting organic acids (OA's) from gastric digestion. Panel A illustrates an encapsulated product, which contains 100% of the active ingredient disposed on the inside of a protective barrier. Panel B illustrates a matrix-embedded composition of the invention. As illustrated in the schematic, the embedded OA's are disposed on the surface or within the matrix.

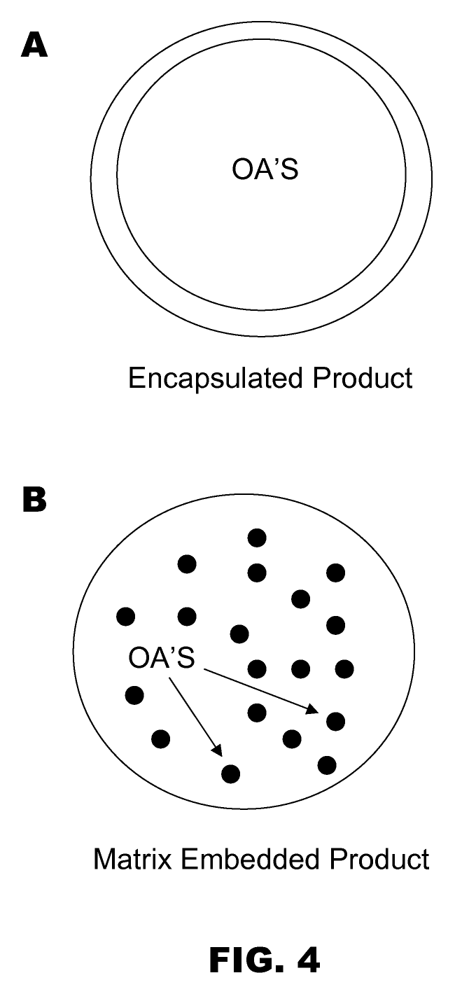

### DETAILED DESCRIPTION OF THE INVENTION

The invention provides matrix-embedded compositions having organic acids and fatty acids. Because the compositions of the invention are embedded within a matrix, they are generally resistant to degradation in the acidic stomach. Once the matrix-embedded compositions enter the small intestine, however, intestinal enzymes, such as lipases and esterases, may hydrolyze the composition, causing the release of intact organic acid and fatty acids from the matrix. In addition to providing nutritional benefits, the organic acids and fatty acids may also provide antimicrobial activity. As illustrated in the Examples, administration of the matrix-embedded compositions to monogastric animals generally increases overall weight gain and feed efficiency compared to matrix-embedded compositions having only organic acids.

I. Matrix-Embedded Compositions

One aspect of the invention provides a composition that is embedded in a matrix. Generally speaking, the composition comprises an organic acid and a fatty acid. Suitable examples of organic acids, fatty acids, and matrices are detailed below.

(a) Matrix

A variety of compounds or compositions are suitable for use as a matrix. In the context of the invention, the term “matrix” is used in its broadest sense and includes any of a variety of compounds or compositions to which a composition comprising an organic acid and a fatty acid may be embedded. In an exemplary embodiment, the matrix will comprise a fat source. Generally speaking, a suitable matrix is one that can be embedded with a relatively high density of a composition comprising an organic acid and a fatty acid. In the context of the invention, the term “embedded” generally means that the fatty acids and organic acids are disposed on the surface of or within the matrix. The term “matrix-embedded” does not include encapsulated products. Encapsulated products typically contain 100% of the active agent (e.g., organic acid or fatty acid) disposed inside of a protective coating or barrier.

In one embodiment, the matrix material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum.

In another embodiment, the matrix material may comprise a protein. Suitable proteins include, but are not limited to, gelatin, casein, collagen, whey proteins, soy proteins, rice protein, and corn proteins.

In still another embodiment, the matrix material may comprise an edible wax. Edible waxes may be derived from mammals, insects, or plants. Non-limiting examples include beeswax, lanolin, bayberry wax, carnauba wax, and rice bran wax. The matrix material may also comprise a mixture of biopolymers. As an example, the matrix material may comprise a mixture of a polysaccharide and a fat.

In yet another embodiment, the matrix material may comprise a semi-synthetic polymer. Semi-synthetic polymers include, but are not limited to, semi-synthetic celluloses and semi-synthetic starches. The semi-synthetic celluloses include methylcellulose, ethylcellulose, hydroxyethylcellulose, carboxymethylcellulose, hydroxypropylcellulose, hydroxypropylmethylcellulose, sulfonated cellulose, cellulose acetate, cellulose acetate phthalate, cellulose acetate trimelitate, cellulose ethyl phthalate, and viscose. Suitable semi-synthetic starches include water-soluble starch, carboxymethylated starch, dialdehyde starch, hydrophobically modified starch, oxidized starch, etherified starch, and esterified starch.

In an exemplary embodiment, the matrix will comprise a lipid material. The lipid material can be derived from animal or vegetable origins, such as, for example, coconut oil, wheat germ oil, corn oil, rapeseed oil, palm oil, soybean oil, cottonseed oil, canola oil, olive oil, safflower oil, sunflower oil, and poultry fat. Generally, the lipid is preferably hydrogenated, and can be saturated or partially saturated. Examples of suitable lipid materials include, but are not limited to, monoglycerides, diglycerides, fatty acids, esters of fatty acids, phospholipids, salts thereof, and combinations thereof.

Monoglycerides and diglycerides can be formed naturally in a biological system, as well as by partial or complete hydrolysis of triglycerides and distillation in commercial manufacturing. These methods are known to those skilled in the art. Monoglycerides, also known as monoacylglycerols, are molecules made up of a glycerol and a fatty acid bound as an ester. Diglycerides (i.e., diacylglycerols) are molecules made up of a glycerol and two fatty acids, each fatty acid is bound to the glycerol as an ester. Depending upon the nature of the fatty acid molecule(s) contained in the mono- or diglyceride, the properties of the lipid material may vary.

Phospholipids can be, for example, monoacyl and diacyl phospholipids. Examples of phospholipids include, but are not limited to, phosphatidic acid, phosphatidyl choline, phosphatidyl ethanolamine, phosphatidyl inositol, phosphatidyl serine, phosphatidyl glycerol, and diphosphatidyl glycerol.

The fatty acids can have a carbon chain length of about 4 carbon atoms to about 24 carbon atoms. In an exemplary embodiment, the fatty acid will have a carbon chain length from about 12 carbon atoms to about 22 carbon atoms. The fatty acid can be saturated or unsaturated (e.g., partially saturated), in free form or esterified to glycerol. Examples of such fatty acids include, but are not limited to lauric acid, myristic acid, palmitic acid, stearic acid, palmitoleic acid, oleic acid, ricinoleic acid, and linoleic acid.

The fatty acid esters can be mono- or diglycerol esters formed from fatty acids having from 4 to 24 carbon atoms, such as for example glyceryl distearate, glyceryl monostearate, glyceryl dipalmitate, glyceryl monopalmitate, glyceryl dilaurate, glyceryl didocosanoate, glyceryl monodocosanoate, glyceryl monocaprate, glyceryl dicaprate, glyceryl monomyristate, glyceryl dimyristate, glyceryl monodecenoate, or glyceryl didecenoate.

The lipid material is preferably a food grade lipid material. Some examples of food grade lipid materials include sorbitan monostearates, sorbitan tristearates, calcium stearoyl lactylates, and calcium stearoyl lactylates. Examples of food grade fatty acid esters that are lipid materials include acetic acid esters of mono- and diglycerides, citric acid esters of mono- and di-glycerides, lactic acid esters of mono- and di-gylcerides, polyglycerol esters of fatty acids, propylene glycol esters of fatty acids, and diacetyl tartaric acid esters of mono- and diglycerides.

The concentration of matrix material comprising the composition can and will vary without departing from the scope of the invention. The matrix may comprise from about 1% to about 99% by weight of the composition. In another embodiment, the matrix will comprise from about 25% to about 75% by weight of the composition. In still another embodiment, the matrix will comprise from about 40% to about 60% by weight of the composition. In additional embodiments, the matrix may comprise about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or greater than about 95% by weight of the composition.

(b) Organic Acids

The composition of the invention includes at least one organic acid. A variety of suitable organic acids may be utilized in the compositions of the invention. Typically, the organic acid will be a carboxylic acid or a substituted carboxylic acid having acidic properties. In an exemplary embodiment, the organic acid may also provide antimicrobial activity. The organic acid may be a monocarboxylic acid having a straight chain or it may be branched; it may be saturated or unsaturated.

A variety of organic acids comprised of carboxylic acids are suitable. In one embodiment, the organic acid may contain from about two to about twenty-five carbon atoms. In another embodiment, the organic acid may have from about three to about twenty-two carbon atoms. In a further embodiment, the organic acid may contain from about three to about twelve carbon atoms. In yet another embodiment, the organic acid may contain from about eight to about twelve carbon atoms. In still another embodiment, the organic acid may contain from about two to about six carbon atoms. Suitable organic acids, by way of non-limiting example, include formic acid, acetic acid, propionic acid, butanoic acid, benzoic acid, lactic acid, malic acid, tartaric acid, mandelic acid, citric acid, fumaric acid, sorbic acid, boric acid, succinic acid, adipic acid, glycolic acid, cinnamaldehyde, and glutaric acid.

Salts of organic acids comprising carboxylic acids are also suitable for certain embodiments. Representative suitable salts include the ammonium, magnesium, calcium, lithium, sodium, potassium, selenium, iron, copper, and zinc salts of organic acids. In one embodiment, the organic acid is an ammonium, magnesium, calcium, lithium, sodium, potassium, selenium, iron, copper, or zinc salt of formic acid. In another embodiment, the organic acid is an ammonium, magnesium, calcium, lithium, sodium, potassium, selenium, iron, copper, or zinc salt of acetic acid. In yet another embodiment, the organic acid is an ammonium, magnesium, calcium, lithium, sodium, potassium, selenium, iron, copper, or zinc salt of propionic acid. In an additional embodiment, the organic acid is an ammonium, magnesium, calcium, lithium, sodium, potassium, selenium, iron, copper, or zinc salt of butanoic acid. In a further embodiment, the organic acid is an ammonium, magnesium, calcium, lithium, sodium, potassium, selenium, iron, copper, or zinc salt of benzoic acid. In still another embodiment, the organic acid is an ammonium, magnesium, calcium, lithium, sodium, potassium, selenium, iron, copper, or zinc salt of lactic acid. In yet another embodiment, the organic acid is an ammonium, magnesium, calcium, lithium, sodium, potassium, selenium, iron, copper, or zinc salt of malic acid. In still another embodiment, the organic acid is an ammonium, magnesium, calcium, lithium, sodium, potassium, selenium, iron, copper, or zinc salt of tartaric acid. In a further embodiment, the organic acid is an ammonium, magnesium, calcium, lithium, sodium, potassium, selenium, iron, copper, or zinc salt of mandelic acid. In yet another embodiment, the organic acid is an ammonium, magnesium, calcium, lithium, sodium, potassium, selenium, iron, copper, or zinc salt of citric acid. In an additional embodiment, the organic acid is an ammonium, magnesium, calcium, lithium, sodium, potassium, selenium, iron, copper, or zinc salt of fumaric acid. In an additional embodiment, the organic acid is an ammonium, magnesium, calcium, lithium, sodium, potassium, selenium, iron, copper, or zinc salt of sorbic acid. In another embodiment, the organic acid is an ammonium, magnesium, calcium, lithium, sodium, potassium, selenium, iron, copper, or zinc salt of boric acid. In yet another embodiment, the organic acid is an ammonium, magnesium, calcium, lithium, sodium, potassium, selenium, iron, copper, or zinc salt of succinic acid. In another embodiment, the organic acid is an ammonium, magnesium, calcium, lithium, sodium, potassium, selenium, iron, copper, or zinc salt of adipic acid. In yet another embodiment, the organic acid is an ammonium, magnesium, calcium, lithium, sodium, potassium, selenium, iron, copper, or zinc salt of glycolic acid. In an additional embodiment, the organic acid is an ammonium, magnesium, calcium, lithium, sodium, potassium, selenium, iron, copper, or zinc salt of glutaric acid.

Alternatively, the organic acid may be comprised of a substituted carboxylic acid. A substituted carboxylic acid generally has the same features as those detailed above for carboxylic acids, but the hydrocarbyl chain has been modified such that it is branched, is part of a ring structure, or contains some other substitution. In one embodiment, the substituted carboxylic acid may contain one or more additional carboxyl groups. Saturated dicarboxylic acids include malonic acid, succinic acid, glutaric acid, and adipic acid, and unsaturated dicarboxylic acids include maleic acid and fumaric acid. In another embodiment, the substituted carboxylic acid may contain one or more hydroxyl groups. A substituted carboxylic acid with a hydroxyl group on the alpha carbon, i.e., the carbon adjacent to the carboxyl carbon, is generally called a α-hydroxy carboxylic acid. Examples of suitable α-hydroxy carboxylic acids include glycolic acid, lactic acid, malic acid, and tartaric acid. In an alternate embodiment, the substituted carboxylic acid may contain one or more carbonyl groups. In yet another embodiment, the substituted carboxylic acid may contain an amino group on the alpha carbon, i.e., is an α-amino acid. In one embodiment, the α-amino acid may be one of the twenty standard amino acids or derivatives thereof. In another embodiment, the α-amino acid may be an essential α-amino acid selected from the group consisting of arginine, histidine, isoleucine, leucine, lysine, methionine, phenylalanine, threonine, tryptophan, and valine. Salts of organic acids comprising substituted carboxylic acids are also suitable for certain embodiments. Representative suitable salts include the ammonium, magnesium, calcium, lithium, sodium, potassium, selenium, iron, copper, and zinc salts of organic acids comprising substituted carboxylic acids.

In yet another embodiment, the organic acid may be a compound having Formula (I):

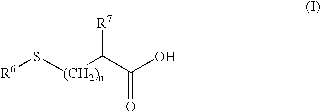

wherein:
n is an integer from 0 to 2;
R⁶ is an alkyl group having from one to four carbon atoms;
R⁷ is selected from the group consisting of hydroxyl, amino, and —OCOR⁸ or —NHCOR⁸; and
R⁸ is an organic acid derivative.

In an exemplary embodiment for compounds having Formula (I), R⁶ is methyl or ethyl; R⁷ is hydroxyl or amino; and n is 0 to 2.

Salts of compounds having Formula (I) are also suitable for certain embodiments. Representative salts of the compound of Formula (I) include the ammonium, magnesium, calcium, lithium, sodium, potassium, selenium, iron, copper, and zinc salts. In a preferred embodiment, the compound of Formula (I) is in the form of the calcium salt. Representative amides include methylamide, dimethylamide, ethylmethylamide, butylamide, dibutylamide, butylmethylamide, alkyl ester of N-acyl methionates (e.g., alkyl N-acetyl methionates. Representative esters include the methyl, ethyl, n-propyl, isopropyl, butyl esters, namely n-butyl, sec-butyl, isobutyl, and t-butyl esters, pentyl esters and hexyl esters, especially n-pentyl, isopentyl, n-hexyl and isohexyl esters.

In various preferred embodiments, the compound of Formula (I) is 2-hydroxy-4-(methylthio)butanoic acid (HMTBA) or a salt, amide or ester thereof, such as any of those detailed above. In still more preferred embodiments, the compound of Formula (I) is HMTBA.

The concentration of organic acid comprising the composition can and will vary without departing from the scope of the invention. The organic acid may comprise from about 1% to about 99% by weight of the composition. In another embodiment, the organic acid will comprise from about 25% to about 75% by weight of the composition. In still another embodiment, the organic acid will comprise from about 40% to about 60% by weight of the composition. In additional embodiments, the organic acid may comprise about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or greater than about 95% by weight of the composition.

(c) Fatty Acids

The composition of the invention also includes at least one fatty acid. The fatty acid may have a straight chain or it may be branched; it may be saturated or unsaturated. The fatty acid may also be bound to other molecules, such as in triglycerides or phospholipids. Alternatively, the fatty acid may be an uncombined or free fatty acid. In this context, a “free” fatty acid is not attached to another molecule.

In certain embodiment, the fatty acid is a saturated aliphatic compound having from four to twenty-two carbon atoms. In an exemplary embodiment, the fatty acid will comprise from four to twelve carbon atoms. By way of non-limiting example, the fatty acid may be butanoic acid (C4:0), hexanoic acid (C6:0), octanoic acid (C8:0), decanoic acid (C10:0), dodecanoic acid (C12:0), tetradecanoic acid (C14:0), hexadecanoic acid (C16:0), octadecanoic acid (C18:0), eicosanoic acid (C20:0), and docosanoic acid (C22:0). In an exemplary embodiment, the fatty acid is selected from octanoic acid, decanoic acid, and dodecanoic acid. In another exemplary embodiment, the fatty acid is a mixture of octanoic acid and decanoic acid. In another exemplary embodiment, the fatty acid is a mixture of hexanoic acid, octanoic acid, decanoic acid, and dodecanoic acid.

Alternatively, the fatty acid may be an unsaturated aliphatic compound. Suitable examples of unsaturated fatty acids include a hexanoic acid with two double bonds (C6:2), myristoleic acid (i.e., a C₁₄ acid with one double bond (C14:1)), palmitoleic acid (C16:1), oleic acid (C18:1), linoleic acid (C18:2), linolenic (C18:3), gadoleic acid (C20:1), arachidonic acid (C20:4), eicosapentaenoic acid (C20:5), docosahexaenoic acid (C22:6), and erucic acid (C22:1).

The concentration of fatty acid comprising the composition can and will vary without departing from the scope of the invention. The fatty acid may comprise from about 0.01% to about 10% by weight of the composition. In another embodiment, the fatty acid will comprise from about 0.05% to about 5% by weight of the composition. In still another embodiment, the fatty acid will comprise from about 0.1% to about 1% by weight of the composition. In additional embodiments, the fatty acid may comprise about 0.05%, about 0.1%, about 0.2%, about 0.3%, about 0.4%, about 0.5%, about 0.6%, about 0.7%, about 0.8%, about 0.9%, or greater than about 1% by weight of the composition.

(d) Combinations of Organic Acids and Fatty Acids

Any of the organic acids detailed herein or otherwise known in the art may be combined with any of the fatty acids detailed herein or otherwise known in the art to form a composition of the invention. As will be appreciated by a skilled artisan, a composition of the invention may include from one to several organic acid(s) combined with from one to several fatty acids and the composition may then be embedded in any of the matrices detailed herein. Suitable examples of combinations of organic acids and fatty acids are detailed in Table A.

TABLE A

| Organic Acid | Fatty Acid |
|---|---|
| Formic acid | Butanoic acid |
| Formic acid | Hexanoic acid |
| Formic acid | Octanoic acid |
| Formic acid | Decanoic acid |
| Formic acid | Dodecanoic acid |
| Formic acid | Tetradecanoic acid |
| Formic acid | Hexadecanoic acid |
| Formic acid | Octadecanoic acid |
| Formic acid | Eicosanoic acid |
| Formic acid | Docosanoic acid |
| Formic acid | Hexanoic acid |
| Formic acid | Myristoleic acid |
| Formic acid | Palmitoleic acid |
| Formic acid | Oleic acid |
| Formic acid | Linoleic acid |
| Formic acid | Linolenic |
| Formic acid | Gadoleic acid |
| Formic acid | Arachidonic acid |
| Formic acid | Eicosapentaenoic acid |
| Formic acid | Docosahexaenoic acid |
| Formic acid | Erucic acid |
| Acetic acid | Butanoic acid |
| Acetic acid | Hexanoic acid |
| Acetic acid | Octanoic acid |
| Acetic acid | Decanoic acid |
| Acetic acid | Dodecanoic acid |
| Acetic acid | Tetradecanoic acid |
| Acetic acid | Hexadecanoic acid |
| Acetic acid | Octadecanoic acid |
| Acetic acid | Eicosanoic acid |
| Acetic acid | Docosanoic acid |
| Acetic acid | Hexanoic acid |
| Acetic acid | Myristoleic acid |
| Acetic acid | Palmitoleic acid |
| Acetic acid | Oleic acid |
| Acetic acid | Linoleic acid |
| Acetic acid | Linolenic |
| Acetic acid | Gadoleic acid |
| Acetic acid | Arachidonic acid |
| Acetic acid | Eicosapentaenoic acid |
| Acetic acid | Docosahexaenoic acid |
| Acetic acid | Erucic acid |
| Propionic acid | Butanoic acid |
| Propionic acid | Hexanoic acid |
| Propionic acid | Octanoic acid |
| Propionic acid | Decanoic acid |
| Propionic acid | Dodecanoic acid |
| Propionic acid | Tetradecanoic acid |
| Propionic acid | Hexadecanoic acid |
| Propionic acid | Octadecanoic acid |
| Propionic acid | Eicosanoic acid |
| Propionic acid | Docosanoic acid |
| Propionic acid | Hexanoic acid |
| Propionic acid | Myristoleic acid |
| Propionic acid | Palmitoleic acid |
| Propionic acid | Oleic acid |
| Propionic acid | Linoleic acid |
| Propionic acid | Linolenic |
| Propionic acid | Gadoleic acid |
| Propionic acid | Arachidonic acid |
| Propionic acid | Eicosapentaenoic acid |
| Propionic acid | Docosahexaenoic acid |
| Propionic acid | Erucic acid |
| Butanoic acid | Butanoic acid |
| Butanoic acid | Hexanoic acid |
| Butanoic acid | Octanoic acid |
| Butanoic acid | Decanoic acid |
| Butanoic acid | Dodecanoic acid |
| Butanoic acid | Tetradecanoic acid |
| Butanoic acid | Hexadecanoic acid |
| Butanoic acid | Octadecanoic acid |
| Butanoic acid | Eicosanoic acid |
| Butanoic acid | Docosanoic acid |
| Butanoic acid | Hexanoic acid |
| Butanoic acid | Myristoleic acid |
| Butanoic acid | Palmitoleic acid |
| Butanoic acid | Oleic acid |
| Butanoic acid | Linoleic acid |
| Butanoic acid | Linolenic |
| Butanoic acid | Gadoleic acid |
| Butanoic acid | Arachidonic acid |
| Butanoic acid | Eicosapentaenoic acid |
| Butanoic acid | Docosahexaenoic acid |
| Butanoic acid | Erucic acid |
| Benzoic acid | Butanoic acid |
| Benzoic acid | Hexanoic acid |
| Benzoic acid | Octanoic acid |
| Benzoic acid | Decanoic acid |
| Benzoic acid | Dodecanoic acid |
| Benzoic acid | Tetradecanoic acid |
| Benzoic acid | Hexadecanoic acid |
| Benzoic acid | Octadecanoic acid |
| Benzoic acid | Eicosanoic acid |
| Benzoic acid | Docosanoic acid |
| Benzoic acid | Hexanoic acid |
| Benzoic acid | Myristoleic acid |
| Benzoic acid | Palmitoleic acid |
| Benzoic acid | Oleic acid |
| Benzoic acid | Linoleic acid |
| Benzoic acid | Linolenic |
| Benzoic acid | Gadoleic acid |
| Benzoic acid | Arachidonic acid |
| Benzoic acid | Eicosapentaenoic acid |
| Benzoic acid | Docosahexaenoic acid |
| Benzoic acid | Erucic acid |
| Lactic acid | Butanoic acid |
| Lactic acid | Hexanoic acid |
| Lactic acid | Octanoic acid |
| Lactic acid | Decanoic acid |
| Lactic acid | Dodecanoic acid |
| Lactic acid | Tetradecanoic acid |
| Lactic acid | Hexadecanoic acid |
| Lactic acid | Octadecanoic acid |
| Lactic acid | Eicosanoic acid |
| Lactic acid | Docosanoic acid |
| Lactic acid | Hexanoic acid |
| Lactic acid | Myristoleic acid |
| Lactic acid | Palmitoleic acid |
| Lactic acid | Oleic acid |
| Lactic acid | Linoleic acid |
| Lactic acid | Linolenic |
| Lactic acid | Gadoleic acid |
| Lactic acid | Arachidonic acid |
| Lactic acid | Eicosapentaenoic acid |
| Lactic acid | Docosahexaenoic acid |
| Lactic acid | Erucic acid |
| Malic acid | Butanoic acid |
| Malic acid | Hexanoic acid |
| Malic acid | Octanoic acid |
| Malic acid | Decanoic acid |
| Malic acid | Dodecanoic acid |
| Malic acid | Tetradecanoic acid |
| Malic acid | Hexadecanoic acid |
| Malic acid | Octadecanoic acid |
| Malic acid | Eicosanoic acid |
| Malic acid | Docosanoic acid |
| Malic acid | Hexanoic acid |
| Malic acid | Myristoleic acid |
| Malic acid | Palmitoleic acid |
| Malic acid | Oleic acid |
| Malic acid | Linoleic acid |
| Malic acid | Linolenic |
| Malic acid | Gadoleic acid |
| Malic acid | Arachidonic acid |
| Malic acid | Eicosapentaenoic acid |
| Malic acid | Docosahexaenoic acid |
| Malic acid | Erucic acid |
| Tartaric acid | Butanoic acid |
| Tartaric acid | Hexanoic acid |
| Tartaric acid | Octanoic acid |
| Tartaric acid | Decanoic acid |
| Tartaric acid | Dodecanoic acid |
| Tartaric acid | Tetradecanoic acid |
| Tartaric acid | Hexadecanoic acid |
| Tartaric acid | Octadecanoic acid |
| Tartaric acid | Eicosanoic acid |
| Tartaric acid | Docosanoic acid |
| Tartaric acid | Hexanoic acid |
| Tartaric acid | Myristoleic acid |
| Tartaric acid | Palmitoleic acid |
| Tartaric acid | Oleic acid |
| Tartaric acid | Linoleic acid |
| Tartaric acid | Linolenic |
| Tartaric acid | Gadoleic acid |
| Tartaric acid | Arachidonic acid |
| Tartaric acid | Eicosapentaenoic acid |
| Tartaric acid | Docosahexaenoic acid |
| Tartaric acid | Erucic acid |
| Mandelic acid | Butanoic acid |
| Mandelic acid | Hexanoic acid |
| Mandelic acid | Octanoic acid |
| Mandelic acid | Decanoic acid |
| Mandelic acid | Dodecanoic acid |
| Mandelic acid | Tetradecanoic acid |
| Mandelic acid | Hexadecanoic acid |
| Mandelic acid | Octadecanoic acid |
| Mandelic acid | Eicosanoic acid |
| Mandelic acid | Docosanoic acid |
| Mandelic acid | Hexanoic acid |
| Mandelic acid | Myristoleic acid |
| Mandelic acid | Palmitoleic acid |
| Mandelic acid | Oleic acid |
| Mandelic acid | Linoleic acid |
| Mandelic acid | Linolenic |
| Mandelic acid | Gadoleic acid |
| Mandelic acid | Arachidonic acid |
| Mandelic acid | Eicosapentaenoic acid |
| Mandelic acid | Docosahexaenoic acid |
| Mandelic acid | Erucic acid |
| Citric acid | Butanoic acid |
| Citric acid | Hexanoic acid |
| Citric acid | Octanoic acid |
| Citric acid | Decanoic acid |
| Citric acid | Dodecanoic acid |
| Citric acid | Tetradecanoic acid |
| Citric acid | Hexadecanoic acid |
| Citric acid | Octadecanoic acid |
| Citric acid | Eicosanoic acid |
| Citric acid | Docosanoic acid |
| Citric acid | Hexanoic acid |
| Citric acid | Myristoleic acid |
| Citric acid | Palmitoleic acid |
| Citric acid | Oleic acid |
| Citric acid | Linoleic acid |
| Citric acid | Linolenic |
| Citric acid | Gadoleic acid |
| Citric acid | Arachidonic acid |
| Citric acid | Eicosapentaenoic acid |
| Citric acid | Docosahexaenoic acid |
| Citric acid | Erucic acid |
| Fumaric acid | Butanoic acid |
| Fumaric acid | Hexanoic acid |
| Fumaric acid | Octanoic acid |
| Fumaric acid | Decanoic acid |
| Fumaric acid | Dodecanoic acid |
| Fumaric acid | Tetradecanoic acid |
| Fumaric acid | Hexadecanoic acid |
| Fumaric acid | Octadecanoic acid |
| Fumaric acid | Eicosanoic acid |
| Fumaric acid | Docosanoic acid |
| Fumaric acid | Hexanoic acid |
| Fumaric acid | Myristoleic acid |
| Fumaric acid | Palmitoleic acid |
| Fumaric acid | Oleic acid |
| Fumaric acid | Linoleic acid |
| Fumaric acid | Linolenic |
| Fumaric acid | Gadoleic acid |
| Fumaric acid | Arachidonic acid |
| Fumaric acid | Eicosapentaenoic acid |
| Fumaric acid | Docosahexaenoic acid |
| Fumaric acid | Erucic acid |
| Sorbic acid | Butanoic acid |
| Sorbic acid | Hexanoic acid |
| Sorbic acid | Octanoic acid |
| Sorbic acid | Decanoic acid |
| Sorbic acid | Dodecanoic acid |
| Sorbic acid | Tetradecanoic acid |
| Sorbic acid | Hexadecanoic acid |
| Sorbic acid | Octadecanoic acid |
| Sorbic acid | Eicosanoic acid |
| Sorbic acid | Docosanoic acid |
| Sorbic acid | Hexanoic acid |
| Sorbic acid | Myristoleic acid |
| Sorbic acid | Palmitoleic acid |
| Sorbic acid | Oleic acid |
| Sorbic acid | Linoleic acid |
| Sorbic acid | Linolenic |
| Sorbic acid | Gadoleic acid |
| Sorbic acid | Arachidonic acid |
| Sorbic acid | Eicosapentaenoic acid |
| Sorbic acid | Docosahexaenoic acid |
| Sorbic acid | Erucic acid |
| Boric acid | Butanoic acid |
| Boric acid | Hexanoic acid |
| Boric acid | Octanoic acid |
| Boric acid | Decanoic acid |
| Boric acid | Dodecanoic acid |
| Boric acid | Tetradecanoic acid |
| Boric acid | Hexadecanoic acid |
| Boric acid | Octadecanoic acid |
| Boric acid | Eicosanoic acid |
| Boric acid | Docosanoic acid |
| Boric acid | Hexanoic acid |
| Boric acid | Myristoleic acid |
| Boric acid | Palmitoleic acid |
| Boric acid | Oleic acid |
| Boric acid | Linoleic acid |
| Boric acid | Linolenic |
| Boric acid | Gadoleic acid |
| Boric acid | Arachidonic acid |
| Boric acid | Eicosapentaenoic acid |
| Boric acid | Docosahexaenoic acid |
| Boric acid | Erucic acid |
| Succinic acid | Butanoic acid |
| Succinic acid | Hexanoic acid |
| Succinic acid | Octanoic acid |
| Succinic acid | Decanoic acid |
| Succinic acid | Dodecanoic acid |
| Succinic acid | Tetradecanoic acid |
| Succinic acid | Hexadecanoic acid |
| Succinic acid | Octadecanoic acid |
| Succinic acid | Eicosanoic acid |
| Succinic acid | Docosanoic acid |
| Succinic acid | Hexanoic acid |
| Succinic acid | Myristoleic acid |
| Succinic acid | Palmitoleic acid |
| Succinic acid | Oleic acid |
| Succinic acid | Linoleic acid |
| Succinic acid | Linolenic |
| Succinic acid | Gadoleic acid |
| Succinic acid | Arachidonic acid |
| Succinic acid | Eicosapentaenoic acid |
| Succinic acid | Docosahexaenoic acid |
| Succinic acid | Erucic acid |
| Adipic acid | Butanoic acid |
| Adipic acid | Hexanoic acid |
| Adipic acid | Octanoic acid |
| Adipic acid | Decanoic acid |
| Adipic acid | Dodecanoic acid |
| Adipic acid | Tetradecanoic acid |
| Adipic acid | Hexadecanoic acid |
| Adipic acid | Octadecanoic acid |
| Adipic acid | Eicosanoic acid |
| Adipic acid | Docosanoic acid |
| Adipic acid | Hexanoic acid |
| Adipic acid | Myristoleic acid |
| Adipic acid | Palmitoleic acid |
| Adipic acid | Oleic acid |
| Adipic acid | Linoleic acid |
| Adipic acid | Linolenic |
| Adipic acid | Gadoleic acid |
| Adipic acid | Arachidonic acid |
| Adipic acid | Eicosapentaenoic acid |
| Adipic acid | Docosahexaenoic acid |
| Adipic acid | Erucic acid |
| Glycolic acid | Butanoic acid |
| Glycolic acid | Hexanoic acid |
| Glycolic acid | Octanoic acid |
| Glycolic acid | Decanoic acid |
| Glycolic acid | Dodecanoic acid |
| Glycolic acid | Tetradecanoic acid |
| Glycolic acid | Hexadecanoic acid |
| Glycolic acid | Octadecanoic acid |
| Glycolic acid | Eicosanoic acid |
| Glycolic acid | Docosanoic acid |
| Glycolic acid | Hexanoic acid |
| Glycolic acid | Myristoleic acid |
| Glycolic acid | Palmitoleic acid |
| Glycolic acid | Oleic acid |
| Glycolic acid | Linoleic acid |
| Glycolic acid | Linolenic |
| Glycolic acid | Gadoleic acid |
| Glycolic acid | Arachidonic acid |
| Glycolic acid | Eicosapentaenoic acid |
| Glycolic acid | Docosahexaenoic acid |
| Glycolic acid | Erucic acid |
| Glutaric acid | Butanoic acid |
| Glutaric acid | Hexanoic acid |
| Glutaric acid | Octanoic acid |
| Glutaric acid | Decanoic acid |
| Glutaric acid | Dodecanoic acid |
| Glutaric acid | Tetradecanoic acid |
| Glutaric acid | Hexadecanoic acid |
| Glutaric acid | Octadecanoic acid |
| Glutaric acid | Eicosanoic acid |
| Glutaric acid | Docosanoic acid |
| Glutaric acid | Hexanoic acid |
| Glutaric acid | Myristoleic acid |
| Glutaric acid | Palmitoleic acid |
| Glutaric acid | Oleic acid |
| Glutaric acid | Linoleic acid |
| Glutaric acid | Linolenic |
| Glutaric acid | Gadoleic acid |
| Glutaric acid | Arachidonic acid |
| Glutaric acid | Eicosapentaenoic acid |
| Glutaric acid | Docosahexaenoic acid |
| Glutaric acid | Erucic acid |
| Formic acid and tartaric acid | Octanoic acid |
| Formic acid and tartaric acid | Decanoic acid |
| Formic acid and tartaric acid | Dodecanoic acid |
| Acetic acid citric acid and boric acid | Octanoic acid |
| Acetic acid citric acid and boric acid | Decanoic acid |
| Acetic acid citric acid and boric acid | Dodecanoic acid |
| Propionic acid, benzoic acid, lactic acid | Octanoic acid |
| and malic acid | |
| Propionic acid, benzoic acid, lactic acid | Decanoic acid |
| and malic acid | |
| Propionic acid, benzoic acid, lactic acid | Dodecanoic acid |
| and malic acid | |

In an embodiment, the organic acid is selected from the group consisting of formic acid, acetic acid, propionic acid, butanoic acid, benzoic acid, lactic acid, malic acid, tartaric acid, mandelic acid, citric acid, fumaric acid, sorbic acid, boric acid, succinic acid, adipic acid, glycolic acid, cinnamaldehyde, glutaric acid, and mixtures thereof; and the fatty acid is selected from the group consisting of hexanoic acid, octanoic acid, decanoic acid, dodecanoic acid, and mixtures thereof. In an exemplary embodiment, the organic acid is selected from formic acid, fumaric acid, sorbic acid, benzoic acid, butanoic acid, propionic acid, and mixtures thereof; and the fatty acid is octanoic acid and/or decanoic acid. In an alternative exemplary embodiment, the organic acid comprises calcium formate, sorbic acid, cinnamaldehyde, and benzoic acid; and the fatty acid is octanoic acid and/or decanoic acid. In an alternative of each of the foregoing embodiments, the fatty acid is a mixture of hexanoic acid, octanoic acid, decanoic acid, and dodecanoic acid.

The embedded compositions may have from about 1% to about 99% by weight of organic acids, from about 0.01% to about 10% by weight of fatty acids, and from about 1% to about 99% by weight of matrix. In an alternative embodiment, the embedded compositions may have from about 25% to about 75% by weight of organic acids, from about 0.05% to about 5% by weight of fatty acids, and from about 25% to about 75% by weight of matrix. In still another embodiment, the embedded compositions may have from about 40% to about 60% by weight of organic acids, from about 0.1% to about 1% by weight of fatty acids, and from about 40% to about 60% by weight of matrix.

In one exemplary embodiment, the embedded composition comprises from about 20% to about 30% by weight of calcium formate; from about 10% to about 20% by weight of benzoic acid; from about 5% to about 15% by weight of sorbic acid; about 1% by weight of cinnamaldehyde; about 1% of the fatty acid mixture; and from about 45% to about 55% by weight of the stearic acid (or other matrix material described herein). The fatty acid mixture may comprise from about 0.1% to about 1% by weight of hexanoic acid; from about 45% to about 65% by weight of octanoic acid; from about 30% to about 45% by weight of decanoic acid; and from about 1% to about 3% by weight of dodecanoic acid.

The compositions of the invention may include additional ingredients without departing from the scope of the invention. By way of non-limiting example, the composition may further optionally include one or more of a mixture of natural amino acids, analogs of natural amino acids, such as a hydroxyl analog of methionine (“HMTBA”), vitamins and derivatives thereof, supplemental protein, enzymes, animal drugs, hormones, effective microorganisms, organic acids, preservatives, flavors, and inert fats.

II. Processes for Making Matrix-Embedded Compositions

Another aspect of the invention encompasses processes for making a matrix-embedded composition. Several suitable processes that produce a matrix that includes an organic acid, and a fatty acid may be utilized. Generally speaking, a process of the invention includes heating the matrix, mixing the heated matrix with the organic acid and the fatty acid to form a solution, and solidifying the solution to form a composition embedded in a matrix. Any of the organic acids, fatty acids, and matrices described above may be used. In the following illustration, a lipid matrix is used (e.g., fat source).

By way on non-limiting example, the process may be initiated by heating a fat source in a vessel for a time sufficient to thoroughly liquefy the fat source. The fat source is heated under continuous agitation to a temperature of from about 50° C. to about 80° C. The vessel may be any suitable vessel that includes a heating and agitation means. The liquefied fat source may then be mixed with an organic acid and a fatty acid to form a solution. The process includes mixing from about 40% by weight to about 60% by weight of a fat source with from about 40% by weight to about 60% by weight of organic acid and from about 0.1% by weight to about 1% by weight of a fatty acid.

The organic acid and fatty acid are contacted with the liquefied fat source in a mixing vessel. The solution is then mixed and heated in the vessel until the organic acid and fatty acid are thoroughly dissolved and the solution reaches a temperature of from about 50° C. to about 80° C., preferably 55° C. The vessel may be any suitable vessel that includes a heating and agitation means.

The solution is then fed into a solidification vessel that crystallizes or agglomerates the solution thereby forming the matrix-embedded composition. Preferably, the solidification vessel is a spray tower. A spray tower operates by atomizing the solution, for example with atomizers and/or nozzles, and contacting the solution with a gas at cool or low temperature. As the solution contacts the cool gas, the solution cools to a solidification temperature. Congealing then takes place at a constant temperature during release of the composition's heat of solidification. When no longer in solution, the droplets further cool to give a stable solid composition embedded in a matrix. The solution typically is introduced into the solidification vessel through the top of the vessel so that as the droplets fall onto the cool gas solidification of the solution starts to occur. Generally speaking, the gas used may be any gas suitable to cool and solidify, agglomerate, or crystallize the solution. In one embodiment, the gas is selected from air and an inert gas. In a preferred embodiment, the gas is air. Preferably, the cool gas is at a temperature of from about 5° C. to about 15° C., preferably about 10° C.

At the end of the manufacturing process, the matrix-embedded compositions may be selected so as to have the desired particle size. As such, the vessel may contain at least one screen to separate the desired sized particles. The vessel may alternatively include more than one screen to separate the matrix-embedded composition into several distinctly sized particles. The particles of undesired size may be recycled back into the mixing vessel to reduce waste of materials or it may be discarded.

III. Food, Food Ingredients, and Feed Compositions

Another aspect of the invention provides food, food ingredients, and feed compositions (i.e., animal feed rations) comprising organic acids and fatty acids embedded in a matrix. The matrix-embedded compositions are generally designed to deliver easily absorbable nutrients to the small intestine of the animal. Typically, the matrix-embedded compositions are generally resistant to degradation in the acidic stomach of a monogastric animal or degradation by rumen microorganisms in a ruminant. Once the matrix-embedded compositions enter the small intestine, however, intestinal enzymes, such as lipases and esterases, may hydrolyze the composition, causing the release of the organic acid and fatty acids from the matrix. The intestinal cells may readily absorb the released organic acids and fatty acids.

The matrix-embedded compositions may also provide antimicrobial activity within certain regions of the gastrointestinal tract. As used herein, the term “inhibit” when used in phrases such as “inhibiting bacteria” means any one or more of (a) killing bacteria or mold; (b) any decrease in growth of the bacteria or mold, which may be measured in terms of colony counts; (c) any decrease in the concentration of bacteria or mold; or (d) the inability of bacteria or mold to grow on a particular selection medium. Each of these may be determined, for instance, by comparing the bacterial or fungal colony counts or concentration of bacteria or mold present in the absence of the application of the methods of the present invention with the bacterial or fungal colony counts or concentration of bacteria or mold after application of the methods of the present invention. Generally speaking, application of suitable bactericides or fungicides will show a ten-fold difference in colony counts.

Animals for which the food, food ingredients and/or feed compositions described herein may be provided include humans, ruminants such as dairy cows, lactating dairy cows, dairy calves, beef cattle, sheep, and goats; aquaculture such as fish and crustaceans (including, but not limited to, salmon, shrimp, carp, tilapia and shell fish); livestock such as swine and horses; poultry such as chickens, turkeys, and hatchlings thereof; and companion animals such as dogs and cats. In a particularly preferred embodiment, the animal is a monogastric.

As will be appreciated by a skilled artisan, the concentration of matrix-embedded compositions of the invention in a particular food, food ingredient and/or feed composition can and will vary without departing from the scope of the invention. Generally, the concentration of matrix-embedded compositions is between about 0.01% and about 15% by weight. In various preferred embodiments, the concentration is between 0.01% and about 10% by weight; between 0.02% and about 5% by weight; between 0.03% and about 4% by weight; between 0.04% and about 3% by weight; between about 0.05% and about 0.6% by weight; and between about 0.06% and about 0.5% by weight.

The exact formulation of the above-mentioned animal feed composition is not critical to the present invention. Feed ingredients are selected according to the nutrient requirements of the particular animal for which the feed is intended; these requirements depend, interalia, upon the age and stage of development of the animal, the sex of the animal, and other factors. Feed ingredients may be grouped into eight classes on the basis of their composition and their use in formulating diets: dry forages and roughages; pasture, range plants and forages fed fresh; silages; energy feeds; protein supplements; mineral supplements; vitamin supplements; and additives. See National Research Council (U.S.) Subcommittee on Feed Composition, United States-Canadian Tables of Feed Composition, 3d rev., National Academy Press, pp. 2, 145 (1982). These classes are, to a certain extent, arbitrary, as some feed ingredients could be classified in more than one class. Typically, a feed formulation will also depend upon the costs associated with each ingredient, with the least-expensive composition of ingredients that gives the needed nutrients being the preferred formulation.

By way of non-limiting example, in one embodiment, the animal ration is formulated for swine. The feed formulation will vary for piglets, grower pigs, gestating sows, and lactating sows. Swine feed formulations typically comprise grains (e.g., corn, barley, grain sorghum, oats, soybeans, wheat, etc.), crude proteins (e.g., fish meal, gluten meal, meat meal, soybean meal, tankage, which is the residue that remains after rendering fat in a slaughterhouse, etc.), crude fat (e.g., fish oils, vegetable oils, animal fats, yellow grease, etc.), supplemental amino acids (e.g., lysine, methionine or methionine analogs, etc), vitamins, minerals, mycotoxin inhibitors, antifungal agents, and pharma/nutriceuticals.

In another embodiment, the animal ration is formulated for aquatic animals. As appreciated by a skilled aquaculturist, the feed formulation depends upon the organism being cultured and the developmental stage of the organism. Typical aquaculture preparations contain energy sources, e.g., protein from animal blood meal, meat and bone meal, poultry meal, crab meal, fish meal, shrimp meal, squid meal, and krill; protein/carbohydrates from plants (e.g., alginates, canola, corn, corn gluten, cottonseed meal, kelp meal, molasses, legumes, peanut meal, rice, soybeans, soy protein concentrate, soybean meal, wheat, and wheat gluten); and oils (e.g., fish oil, vegetable oil). The feed preparation may be further supplemented with amino acids (e.g., arginine, histidine, isoleucine, lysine, methionine, phenylalanine, threonine, tryptophan, and valine); vitamins, minerals, enzymes, mycotoxin inhibitors, ammonia binders (e.g., botanical binders, clay mineral binders), emulsifiers, carotenoids, sterols, flavor enhancers, nutriceuticals, immunostimulants, and probiotics.

In another embodiment, the animal feed ration is formulated for poultry. As noted above, feed formulations depend in part upon the age and stage of development of the animal to be fed. Leeson and Summers (Nutrition of the Chicken, 4^{th} ed., pp. 502-510, University Books 2001)) describe several representative poultry diets for pullets, layers, broilers and broiler breeders. For example, most chicken diets contain energy concentrates such as corn, oats, wheat, barley, or sorghum; protein sources such as soybean meal, other oilseed meals (e.g., peanut, sesame, safflower, sunflower, etc.), cottonseed meal, animal protein sources (meat and bone meal, dried whey, fish meal, etc.), grain legumes (e.g., dry beans, field peas, etc.), and alfalfa; and vitamin and mineral supplements, if necessary (for instance, meat and bone meal is high in calcium and phosphorous, and thus these minerals do not need to be supplemented in a feed ration containing meat and bone meal).

In another embodiment, the animal ration is formulated for a ruminant animal. The nutrient and energy content of many common ruminant feed ingredients have been measured and are available to the public. The National Research Council has published books that contain tables of common ruminant feed ingredients and their respective measured nutrient and energy content. Additionally, estimates of nutrient and maintenance energy requirements are provided for growing and finishing cattle according to the weight of the cattle. National Academy of Sciences, Nutrient Requirements of Beef Cattle, Appendix Tables 1-19, 192-214, (National Academy Press, 2000); Nutrient Requirements of Dairy Cattle (2001), each incorporated herein in its entirety. This information can be utilized by one skilled in the art to estimate the nutritional and maintenance energy requirements of cattle with non-functional rumens, such as calves under about 500 lbs in weight, or cattle with functional rumens, such as growing cattle or dairy cattle.

The matrix-embedded compositions may be formulated as liquids, emulsions, or dry or powdered supplements to be added to other foods, such as grains, protein products, and mixtures thereof. The dry feed supplement may be uniformly dispersed throughout a dry or liquid food. Feed compositions may also be provided as aqueous formulations. An aqueous formulation may be a solution or an emulsion. The aqueous formulation may be added directly to the drinking water of an animal or it may be mixed into or applied to a dry or liquid food. The matrix-embedded compositions may be mixed with the other ingredients in the feed, such as the corn, soybean meal, other feed supplements, etc., as the feed is being formulated. Alternatively, the matrix-embedded compositions may be applied to a pre-mixed or pre-pelleted feed.

### DEFINITIONS

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

Unless otherwise indicated, the alkenyl groups described herein are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

Unless otherwise indicated, the alkynyl groups described herein are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “carboxylic acid” used herein refers to organic acids comprising hydrocarbon groups that contain a carboxyl group (COOH). The hydrocarbon moiety consists exclusively of the elements carbon and hydrogen. Carboxylic acids may have straight chains (aliphatic) of hydrocarbyl groups, or they may be aromatic carboxylic acids, as well as some alicyclic carboxylic acids (i.e., both aliphatic and cyclic). Straight chain aliphatic carboxylic acids preferably have 3 to 24 carbons (including the terminal carboxyl carbon). The hydrocarbon chain of an aliphatic carboxylic acid may be saturated (i.e., the carbon atoms have all the hydrogen atoms they can hold) and contain no double bonds between the carbons. Alternatively, the hydrocarbon chain may be unsaturated and contain one or more double bonds between the some of the carbons. Unsaturated carboxylic acids may assume cis or trans configurations, which refer to the orientation of the hydrogen atoms with respect to the double bond. Cis means “on the same side” and trans means “across” or “on the other side”.

An “essential amino acid” is an amino acid that cannot be synthesized by an organism and must be supplied as part of its diet. It is generally recognized that ten amino acids are essential for humans and animals. The essential amino acids are arginine, histidine, isoleucine, leucine, lysine, methionine, phenylalanine, threonine, tryptophan, and valine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, protected hydroxy, acyl, acyloxy, alkoxy, alkenoxy, alkynoxy, aryloxy, halogen, amido, amino, nitro, cyano, thiol, ketals, acetals, esters and ethers.

“HMTBA” stands for 2-hydroxy-4-(methylthio)butanoic acid (sold under the trade name ALIMET® by Novus International, Inc., St. Louis, Mo.).

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “substituted carboxylic acid” used herein refers to substitutions within the hydrocarbyl chain of a straight chain aliphatic carboxylic acid. Hydrocarbyl moieties may be substituted with at least one atom, including the substitution of a carbon atom with a heteroatom such as nitrogen, oxygen, silicon, phosphorous, boron, sulfur, or a halogen atom. Substitutions may also include hydrocarbyl moieties, such as alkyl, alkenyl, alkynyl, and aryl moieties, with these moieties having one to 20 carbon atoms. Other substituted moieties include hydrocarbyloxy, such as acyloxy, alkoxy, alkenoxy, alkynoxy, aryloxy, hydroxy, protected hydroxy, keto, acyl, acyloxy, nitro, amino, amido, cyano, thiol, ketals, acetals, heterocyclo, esters and ethers. Dicarboxylic acids contain an additional carboxyl group at the other end of the molecule. α-Hydroxy acids are another type of substituted carboxylic acid; α-hydroxy acids generally have a hydroxyl group on the alpha carbon atom (i.e., the carbon adjacent to the terminal carbonyl carbon). α-Amino acids, which have an amino group on the alpha carbon, are also substituted carboxylic acids.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, carbocycle, aryl, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters and ethers.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate various iterations of the invention.

### Example 1

### Method of Making Composition

A series of storage bins containing the different organic acid pellets will be positioned and connected to a main mixer bin. Vegetal oil will be warmed in the mixer until it liquefies. Next, the solid materials (acids) will be added to start the mixing process. After the mix has reached a desired pressure and temperature, it will be pumped (using continuous flow) to the top of a spraying tower. There the mix will pass though a nozzles that spray the mix through a column of cool air resulting in the crystallization of the mix into fatty spheres. These spheres will fall to the bottom of the tower due to gravity. While falling, the spheres will cool down. At the bottom of the tower are three separate layers of screens that vary in size. The screens will separate the spheres based on size; only the mid-size product will be kept. The other two sizes will be sent through the process again to avoid wasting materials, and to ensure uniform blending characteristics of the product at the feed mill plant.

### Example 2

### Efficacy of Composition

A composition of the invention was fed to weaned pigs as part of a management program trial. The program was designed to test whether the pigs could maintain a high health status and a daily weight gain while receiving moderated feed. The trial was run at the Research and Demonstration Station of St. Wendelin of the Bingen Institute, Germany. 104 piglets, male/female cross hybrid, were fed for 21 days with one of three different treatments. A total of 8 groups with 13 piglets each were used. The piglets were fed 0.3% (of dry matter) of an embedded composition of the invention, another organic acid composition, or a control diet. The embedded composition of the invention contained calcium formate, benzoic acid, sorbic acid, octanoic acid, decanoic acid, and a matrix that included palm oil. The organic acid composition contained calcium formate, benzoic acid, sorbic acid, and a matrix that included palm oil. The control diet contained no added organic acid.

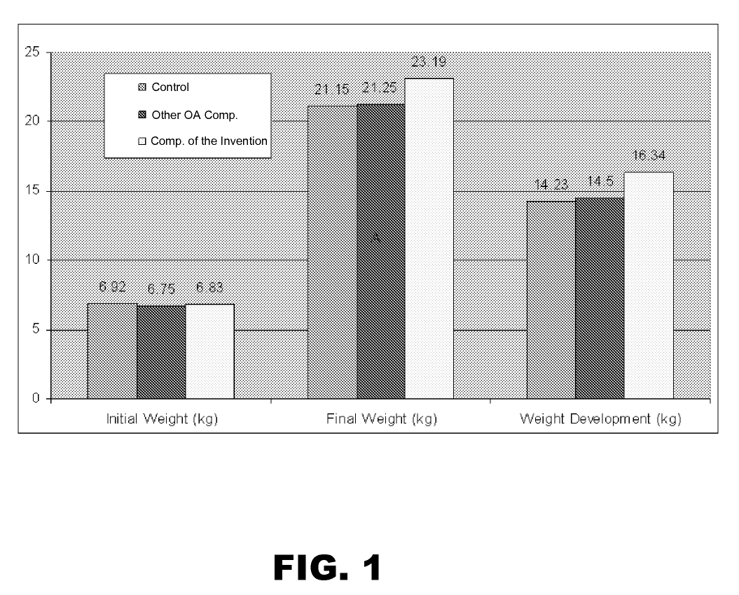

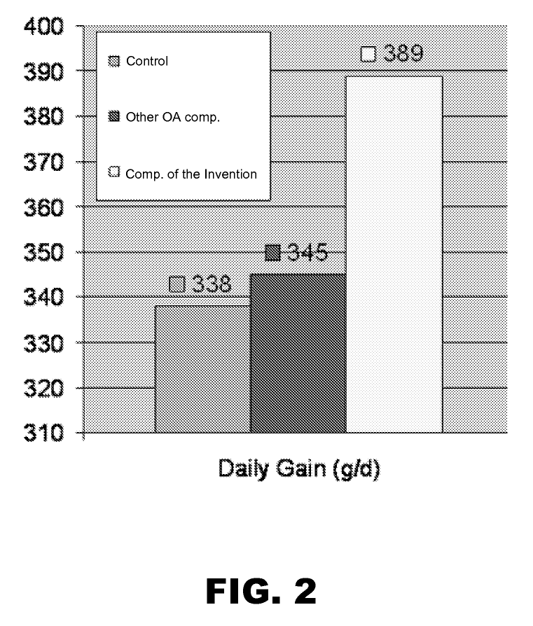

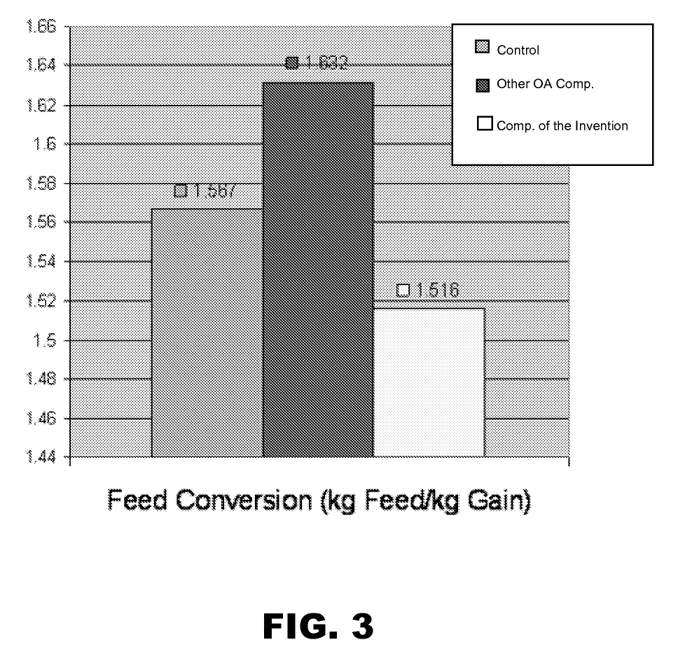

The piglets fed the composition of the invention had a greater final weight gain and a greater weight development than the piglets fed a control diet or another organic acid composition (see FIG. 1). Additionally, the piglets fed the composition of the invention had greater daily weight gain than the piglets fed a control diet or another organic acid composition (see FIG. 2). Furthermore, the piglets fed the composition of the invention had a lower (i.e. more efficient) feed conversion than the piglets fed a control diet or another organic acid composition (see FIG. 3).

What is claimed is:

1. A composition embedded in a lipid matrix, the composition comprising an organic acid and fatty acid having from four to twelve carbon atoms.

2. The composition of claim 1, wherein the lipid matrix comprises a fatty acid having from 12 to 22 carbon atoms.

3. The composition of claim 1, wherein the lipid matrix is selected from the group of vegetable oils consisting of coconut oil, palm oil, cottonseed oil, wheat germ oil, soy oil, olive oil, corn oil, sunflower oil, safflower oil, rapeseed oil, and mixtures thereof.

4. The composition of claim 1, wherein the organic acid is selected from the group consisting of formic acid, cinnamaldehyde, acetic acid, propionic acid, butanoic acid, benzoic acid, lactic acid, malic acid, tartaric acid, mandelic acid, citric acid, fumaric acid, sorbic acid, boric acid, succinic acid, adipic acid, glycolic acid, glutaric acid, and mixtures thereof.

5. The composition of claim 1, wherein the fatty acid is selected from the group consisting of hexanoic acid, octanoic acid, decanoic acid, dodecanoic acid, and mixtures thereof.

6. The composition of claim 1, wherein the organic acid is selected from the group consisting of formic acid, cinnamaldehyde, acetic acid, propionic acid, butanoic acid, benzoic acid, lactic acid, malic acid, tartaric acid, mandelic acid, citric acid, fumaric acid, sorbic acid, boric acid, succinic acid, adipic acid, glycolic acid, glutaric acid, and mixtures thereof; and the fatty acid is selected from the group consisting of hexanoic acid, octanoic acid, decanoic acid, dodecanoic acid, and mixtures thereof.

7. The composition of claim 6, wherein the lipid matrix is selected from the group of vegetable oils consisting of coconut oil, palm oil, cottonseed oil, wheat germ oil, soy oil, olive oil, corn oil, sunflower oil, safflower oil, rapeseed oil, and mixtures thereof.

8. The composition of claim 6, wherein the lipid matrix comprises a fatty acid having from 12 to 22 carbon atoms.

9. The composition of claim 1, wherein the composition comprises from about 25% to about 75% by weight of organic acid, from about 0.05% to 5% by weight of fatty acid; and from about 25% to about 75% by weight of lipid matrix.

10. The composition of claim 1, wherein the composition comprises about 40% to about 60% by weight of organic acid; from about 0.1% to about 1% by weight of fatty acid; and from about 40% to about 60% by weight of lipid matrix.

11. The composition of claim 1, wherein the lipid matrix is stearic acid; the organic acid comprises a mixture of calcium formate, benzoic acid, sorbic acid, and cinnamaldehyde; and the fatty acid comprises a mixture of hexanoic acid, octanoic acid, decanoic acid, and dodecanoic acid.

12. The composition of claim 11, wherein the composition comprises from about 25% to about 75% by weight of the organic acid mixture, from about 0.05% to 5% by weight of the fatty acid mixture; and from about 25% to about 75% by weight of stearic acid.

13. The composition of claim 11, wherein the composition comprises from about 40% to about 60% by weight of the organic acid mixture, from about 0.1% to 1% by weight of the fatty acid mixture; and from about 40% to about 60% by weight of stearic acid.

14. The composition of claim 11, wherein the composition comprises from about 20% to about 30% by weight of calcium formate; from about 10% to about 20% by weight of benzoic acid; from about 5% to about 15% by weight of sorbic acid; about 1% by weight of cinnamaldehyde; about 1% of the fatty acid mixture; and from about 45% to about 55% by weight of the stearic acid.

15. The composition of claim 14, wherein the fatty acid mixture comprises from about 0.1% to about 1% by weight of hexanoic acid; from about 45% to about 65% by weight of octanoic acid; from about 30% to about 45% by weight of decanoic acid; and from about 1% to about 3% by weight of dodecanoic acid.

16. A method for providing an organic acid and a fatty acid having from four to twelve carbon atoms to a monogastric animal, the method comprising administering to the monogastric animal a composition comprising the organic acid and the fatty acid embedded in a lipid matrix, wherein the organic acid and fatty acid are not substantially released from the matrix until the composition enters the monogastric animal's small intestine.

17. The method of claim 16, wherein the monogastric animal is selected from the group consisting of a piglet, a grower pig, a gestating sow, and a lactating sow.

18. The method of claim 16, wherein the organic acid is selected from the group consisting of formic acid, cinnamaldehyde, acetic acid, propionic acid, butanoic acid, benzoic acid, lactic acid, malic acid, tartaric acid, mandelic acid, citric acid, fumaric acid, sorbic acid, boric acid, succinic acid, adipic acid, glycolic acid, glutaric acid, and mixtures thereof; and the fatty acid is selected from the group consisting of hexanoic acid, octanoic acid, decanoic acid, dodecanoic acid, and mixtures thereof.

19. The method of claim 18, wherein the lipid matrix is selected from the group of vegetable oils consisting of coconut oil, palm oil, cottonseed oil, wheat germ oil, soy oil, olive oil, corn oil, sunflower oil, safflower oil, rapeseed oil, and mixtures thereof.

20. The method of claim 18, wherein the lipid matrix comprises a fatty acid having from 12 to 22 carbon atoms.

21. The method of claim 16, wherein the composition comprises from about 25% to about 75% by weight of organic acid, from about 0.05% to 5% by weight of fatty acid; and from about 25% to about 75% by weight of lipid matrix.

22. The method of claim 16, wherein the lipid matrix is stearic acid; the organic acid comprises a mixture of calcium formate, benzoic acid, sorbic acid, and cinnamaldehyde; and the fatty acid comprises a mixture of hexanoic acid, octanoic acid, decanoic acid, and dodecanoic acid.

23. The method of claim 22, wherein the composition comprises from about 25% to about 75% by weight of the organic acid mixture, from about 0.05% to 5% by weight of the fatty acid mixture; and from about 25% to about 75% by weight of stearic acid.

24. The method of claim 22, wherein the composition comprises from about 20% to about 30% by weight of calcium formate; from about 10% to about 20% by weight of benzoic acid; from about 5% to about 15% by weight of sorbic acid; about 1% by weight of cinnamaldehyde; about 1% of the fatty acid mixture; and from about 45% to about 55% by weight of the stearic acid.

25. The method of claim 16, wherein the composition comprises from about 0.1% to about 5% by weight of the monogastric animal's feed ration.

26. The method of claim 16, wherein the monogastric animal has an increased feed efficiency.

27. The method of claim 16, wherein the monogastric animal has an increased rate of gain.

28. A monogastric animal feed ration, the feed ration comprising grain, crude protein, crude fat, and a composition comprising an organic acid and a fatty acid embedded in a lipid matrix.

29. The monogastric animal feed ration of claim 28, wherein the feed ration comprises from about 0.1% to about 5% by weight of the lipid matrix embedded composition.

30. The monogastric animal feed ration of claim 28, wherein the organic acid is selected from the group consisting of formic acid, cinnamaldehyde, acetic acid, propionic acid, butanoic acid, benzoic acid, lactic acid, malic acid, tartaric acid, mandelic acid, citric acid, fumaric acid, sorbic acid, boric acid, succinic acid, adipic acid, glycolic acid, glutaric acid, and mixtures thereof; and the fatty acid is selected from the group consisting of hexanoic acid, octanoic acid, decanoic acid, dodecanoic acid, and mixtures thereof.

31. The monogastric animal feed ration of claim 30, wherein the lipid matrix is selected from the group of vegetable oils consisting of coconut oil, palm oil, cottonseed oil, wheat germ oil, soy oil, olive oil, corn oil, sunflower oil, safflower oil, rapeseed oil, and mixtures thereof.

32. The monogastric animal feed ration of claim 30, wherein the lipid matrix comprises a fatty acid having from 12 to 22 carbon atoms.

33. The monogastric animal feed ration of claim 28, wherein the composition comprises from about 25% to about 75% by weight of organic acid, from about 0.05% to 5% by weight of fatty acid; and from about 25% to about 75% by weight of lipid matrix.

34. The monogastric animal feed ration of claim 28, wherein the lipid matrix is stearic acid; the organic acid comprises a mixture of calcium formate, benzoic acid, sorbic acid, and cinnamaldehyde; and the fatty acid comprises a mixture of hexanoic acid, octanoic acid, decanoic acid, and dodecanoic acid.

35. The monogastric animal feed ration of claim 34, wherein the composition comprises from about 25% to about 75% by weight of the organic acid mixture, from about 0.05% to 5% by weight of the fatty acid mixture; and from about 25% to about 75% by weight of stearic acid.

36. The monogastric animal feed ration of claim 34, wherein the composition comprises from about 20% to about 30% by weight of calcium formate; from about 10% to about 20% by weight of benzoic acid; from about 5% to about 15% by weight of sorbic acid; about 1% by weight of cinnamaldehyde; about 1% of the fatty acid mixture; and from about 45% to about 55% by weight of the stearic acid.
